(19) United States
(12) Plant Patent Application Publication
(10) Pub. No.: US 2005/0097645 P1
(43) Pub. Date: May 5, 2005

(54) Pomegranate tree denominated 'Smith'

(50) PUNICA GRANATUM; SMITH

(76) Inventor: Gregory R. Smith, Visalia, CA (US)

Correspondence Address: Worrel & Worrel;St. Croix Professional Center, Suite 121, 2109 W. Bullard Avenue, Fresno, CA, 93711-1258, US

(21) Appl. No.: 10/699,307

(22) Filed: Oct. 30, 2003

(51) Int. Cl. A01H 5/00

(52) U.S. Cl. PLT/210

(57) ABSTRACT

A new and distinct variety of pomegranate tree which is somewhat remotely similar to the ‘Granada’ pomegranate tree (U.S. Plant Pat. No. 2,618), but from which it is distinguished by producing fruit which are mature for harvesting and shipment approximately September 11 and in which the fruit is of higher acid content when ripe, producing more juice and arils that are moderate red to deep red coloration and softer chewability than those of the fruit of other pomegranate varieties.

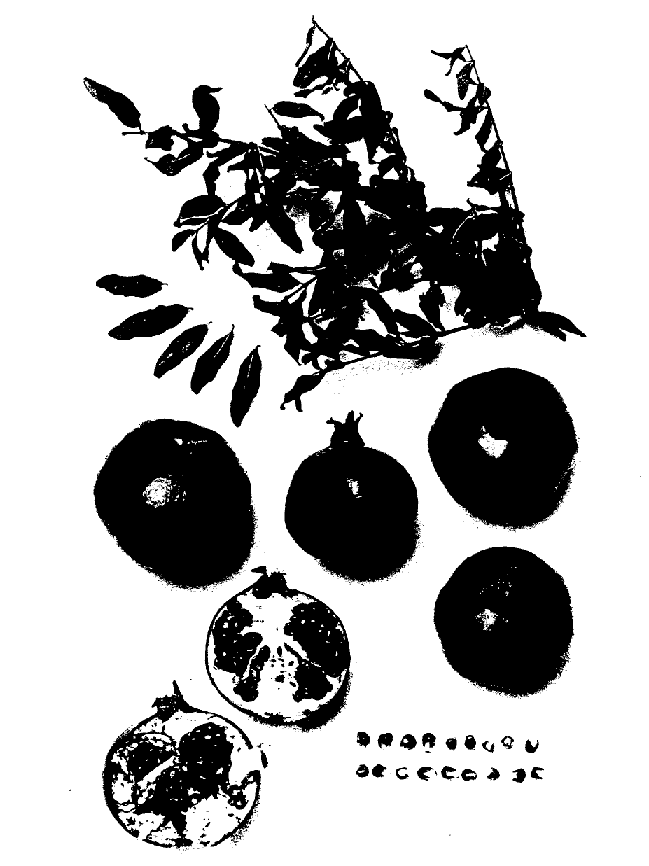

### CROSS-REFERENCE TO RELATED APPLICATIONS

Not Applicable.

### STATEMENT REGARDING FEDERALLY SPONSORED RESEARCH OR DEVELOPMENT

Not Applicable.

### LATIN NAME OF THE GENUS AND SPECIES OF THE PLANT CLAIMED

Punica granatum L.

### VARIETY DENOMINATION

Smith

### BACKGROUND OF THE NEW VARIETY

The present invention relates to a new and distinct variety of pomegranate tree, which will hereinafter be denominated varietally as the ‘Smith’ pomegranate tree, and, more particularly, to a pomegranate tree somewhat similar to the ‘Granada’ pomegranate tree (U.S. Plant Pat. No. 2,618), but which produces fruit with a higher acid content, better flavor and arils which are more chewable, among other distinctions. The new variety of the present invention was discovered as a volunteer seedling growing approximately fifty (50) feet from a ‘Granada’ pomegranate tree commercial planting. The fruit of the variety of the present invention are mature for commercial harvesting and shipment approximately September 11, or three (3) weeks prior to the ‘Wonderful’ pomegranate tree (unpatented) in the San Joaquin Valley of central California. The fruit is distinguished from the ‘Early Foothill’ pomegranate tree, having a redder skin coloration, more juice, higher acid content when mature (1.85%), and softer arils which are easily chewable.

The sales appeal of pomegranates is generally dependent on size and exterior coloration of the fruit. The acceptability of the pomegranate is also influenced by the color of the edible arils, as well as the acid content and flavor of these arils. One other factor that makes the pomegranate more appealing is that it ripens at a time when other fruits of that general type are not available in the marketplace.

The flavor of the fruit of the new variety is directly related to the acid content when fully ripe. The State of California marketing regulations requires that pomegranate fruit may not be marketed until the acid content of the arils, established by titration is 1.85 percent or less. The aril content of this variety reaches this level by approximately September 11. The rind color of the new variety is close to that of the ‘Granada’ pomegranate tree (U.S. Plant Pat. No. 2,621) and is Afghan (P1.5L6) in color, rather than on the pink side. When the fruit of the new variety is cut in cross section, the majority of the arils are found to be of a moderate red to strong red coloration. The texture of the arils of the mature fruit of the new variety is such that they can easily be chewed and softer than the arils of other pomegranate varieties, such as, for example, the ‘Early Wonderful’ pomegranate tree, the ‘Granada’ pomegranate tree and the ‘Early Foothill’ pomegranate tree.

### ORIGIN AND ASEXUAL REPRODUCTION OF THE NEW VARIETY

The present variety of pomegranate tree hereof was discovered by the inventor in his orchard which is located near Visalia in the San Joaquin Valley of central California.

The new variety of pomegranate tree was first observed by the inventor about four (4) years ago in 1996 growing as a volunteer seedling, approximately fifty (50) feet from a commercial planting of ‘Granada’ pomegranate trees. In 1998, the seedling was allowed to produce bloom and fruit. The inventor observed that the rind color of the fruit and maturity date resembled the ‘Granada’ pomegranate tree, but was a heavier juice producer and the arils were significantly softer at maturity. Fruiting bud wood of the new variety has been to asexually reproduce the new variety by grafting on some established ‘Granada’ pomegranate trees. In addition, a number of rooted hard cuttings were made from the original tree. All of the asexually reproduced trees of the new variety have been observed by the inventor to possess fruit and tree characteristics identical to those of the original tree.

### SUMMARY OF THE INVENTION

The ‘Smith’ pomegranate tree is characterized by producing a fruit which is ripe for commercial harvesting and shipment approximately September 11 in the San Joaquin Valley of central California. The new variety is most closely similar to the ‘Granada’ pomegranate tree (U.S. Plant Pat. No. 2,618) and has an exterior color roughly similar to the fruit of that variety, but is distinguishable therefrom in numerous respects including by the aforementioned ripening date.

The new variety is also distinguished from the ‘Dutton’ pomegranate tree (U.S. Plant Pat. No. 4,826). In describing the axillary angle, the angle between the stems and the fruiting branches of the tree measured from 55 degrees to 62 degrees with the average being 60 degrees for the new variety as compared to the ‘Dutton’ pomegranate tree averaging 59.2 degrees.

### BRIEF DESCRIPTION OF THE DRAWING

The accompanying drawing is a color photograph of four whole fruit of the new variety disposed to display the ventral surface, the basal end surface, and the blossom end surface, a fifth fruit of the new variety sectioned to display the interior thereof foliage of the new variety, a number of arils thereof together with a scale to indicate relative size.

### DETAILED BOTANICAL DESCRIPTION

Referring more specifically to the botanical details of this new and distinct variety of pomegranate tree, the following has been observed under the ecological conditions prevailing in the Strathmore area of Tulare County in the San Joaquin Valley of central California. All major color code designations are by reference to the Dictionary of Color, by Maerz and Paul, First Edition, 1930. Common color names are also occasionally employed.

### TREE

Generally: SIZE — Medium, normal for pomegranates. Ten feet high (3.28 meters).
   Vigor.—Good.
   Chilling requirements.—Normal for pomegranate.
   Figure.—Globular/bush.
   Productivity.—Good, 3 to 4 field box/tree.
   Regularity of bearing.—Not alternate but will vary with cultural weather conditions.
Trunk: SIZE.—Multiple, with scaly bark, approximately 10 inch (25.4 cm) diameter and at least 4 feet (1.31 meters) in length.
   Surface texture.—Shaggy or scaly.
   Color.—Brownish Manan (P1. 6 A9).
   Lenticels.—Numbers — Numerous.
   Lenticels.—Size — Small.
Branches: SIZE.—Fruiting wood — 1 inch (2.54 cm) diameter.
   Surface texture.—Smooth.
   Color.—Mature branches — Moss Green (P1. 21 L2) with tan ribs.
   Color.—Immature branches — Brown Sugar (P1. 15 4H).
   Surface texture.—Immature growth — Smooth.
   Lenticels.—Numbers — Numerous. [The angle between the stems and branches of the twigs is closer to the ‘Granada’ pomegranate tree].
   Lenticels.—Size — Small.

### LEAVES

Size: GENERALLY — Small.
   Average length.—45.8 mm (1.80 inches).
   Average width.—15.8 mm (0.62 inches).
   Shape.—Long, oval.
   Color.—Upwardly disposed surface — Mt. Vernon Green (P1. 23 J8).
   Color.—Downwardly disposed surface — Piquant Green (P1. 20 K6).
   Marginal form.—Generally — Smooth.
   Glandular characteristics.—None.
   Petiole.—Size — Short. 2 mm (0.079 inches) to 3 mm (0.12 inches).
   Petiole.—Length — 2 mm (0.079 inches) to 3 mm (0.12 inches).
   Petiole.—Thickness — Very thin. 1.5 mm (0.07 inches).
   Stem glands.—None.
   Petiole.—Short. 2 mm (0.079 inches) to 3 mm (0.12 inches).
   Stipules.—Mostly two.

### FLOWERS

Flower buds.—Generally — Smaller than ‘Wonderful’ variety.
Flower buds.—Size — Length — 10 mm (0.39 inches) to 17 mm (0.76 inches).
Flower buds.—Size — Width — 6 mm (0.23 inches) to 12 mm (0.47 inches).
Flower buds.—Surface Texture — Smooth, slightly waxy.
Flower buds.—Color — Barberry (P1. 5 L7).
Flower buds.—Shape — Slightly elongated 10 mm (0.39 inches) to 17 mm (0.69 inches) in length.
Date of bloom.—May 15^{th} to May 21^{st} (100%).
Flowers.—Number — Single.
Size.—Generally — 37 mm (1.44 inches) to 42 mm (1.65 inches) in diameter.
Petals.—Color — Barberry (P1. 5 L7).
Petals.—Number — Five to seven.
Pistils.—Number — One.
Pistils.—Size — Length — 10 mm (0.39 inches).
Stamens.—Number — Over 60.
Anther.—Small.
Anther.—Color — Tourquet (P1. 4 B8).
Filaments.—Color — Red.
Sepals.—Number — Five to seven.

### FRUIT

Maturity when described: Ripe for commercial harvesting and shipment approximately September 11, three (3) weeks before ‘Wonderful’ pomegranate tree near Strathmore, Calif. in the San Joaquin Valley of central California.
Size: GENERALLY.—Fairly uniform shape, globular.
   Average diameter.—79.25 mm (3.42 inches).
   Average diaxial length.—71.5 mm (2.81 inches).
   Form.—Typical pomegranate shape, prominent crown of calyx segments at apex, symmetrical base, slightly flattened near stem end.
   Form.—Uniformity — Fairly uniform — globular.
   Form.—Symmetry — Individual fruit symmetrical (globular shape).
   Suture.—Generally — Shallow to none.
   Ventral surface.—Generally — N/A.
   Stem.—Very short, 6 mm (0.236 inches) with caliper about the same.
   Stem cavity.—Generally — None.
   Stem.—Generally — Very short.
   Stem.—Caliper — Approximately 6 mm (0.236 inches).
   Rind.—Texture — Hard, corky and tough.
   Tendency to crack.—Late after rains.
   Exterior color.—Afghan (P1. 5 L6).
   Partition color.—Maise (P1. 10 G5).
   Aril.—Color — Azalea (P1. 4 13) to clear.
Skin:
   Texture.—Tough.
   Tendency to crack.—After any fall rains.
   Blush color.—Exterior — Afghan (P1. 5 L6).
Flesh: Flesh Color.—Aril. Moderate red to strong red color.
   Juice production.—Excellent. Plentiful, sweet with typical pomegranate color and grenadine flavor.
   Flavor.—Good. Sweet/grenadine.
   Aroma.—Slight. None.
   Acidity.—1.55% by September 11.
   Rind texture.—Tough.
   Fibers.—Numbers — None.
   Ripening.—Even.
   Eating quality.—Very good, good flavor and good juice production, significant acidity with sugar and distinctive grenadine flavor.
   Base.—Fresh market and juice extract.
Use: Fresh market and juice manufacturer.
Keeping quality: Good.
Resistance to disease: Similar to resistance of ‘Granada’ variety (U.S. Plant Pat. No. 2618).
Harvesting: At maturity — beginning Sep. 11, 2000.

Although the new variety of pomegranate tree possesses the described characteristics noted above as a result of the growing conditions prevailing near Strathmore in the central part of the San Joaquin Valley of California, it is to be understood that variations of the usual magnitude and characteristics incident to changes in growing conditions, irrigation, fertilization, pruning, pest control, climatic variation and the like are to be expected.

1. A new and distinct variety of pomegranate tree substantially as illustrated and described which bears fruit having a deep red rind coloration, arils of color from moderate red to deep red color and with the texture of the arils of the mature fruit easily chewable, being softer than other pomegranate varieties and which has a higher acid content than the fruit of the ‘Granada’ pomegranate tree at maturity and which ripens for harvesting and shipment approximately September 11 in the San Joaquin Valley of central California.
